(19) United States
(12) Patent Application Publication
(10) Pub. No.: US 2021/0259917 A1
(43) Pub. Date: Aug. 26, 2021

(54) APPLICATORS, COMPOSITIONS AND METHODS FOR CLEANSING AN OCULAR AREA

(71) Applicant: OCuSOFT, Inc., Rosenberg, TX (US)

(72) Inventors: Troy Smith, Richmond, TX (US); Suzanne Moon, Richmond, TX (US)

(21) Appl. No.: 17/233,765

(22) Filed: Apr. 19, 2021

Related U.S. Application Data
(63) Continuation-in-part of application No. 16/105,726, filed on Aug. 20, 2018, now Pat. No. 10,980,708.
Application No. 16/105,726 is a division of application No. 14/707,187, filed on May 8, 2015, now Pat. No. 10,076,465.
Application No. 14/707,187 is a continuation-in-part of application No. 14/274,198, filed on May 9, 2014, now abandoned.

(51) Int. Cl. A61H 35/02; A61F 9/00

(52) U.S. Cl. CPC A61H 35/02; A61K 9/0048; A61H 2201/0153; A61F 9/0008

(57) ABSTRACT

A first ocular treatment kit comprises one or more sealed wrappers containing an applicator and a first ocular composition. The applicator includes a shaft and a swab, wherein the swab is coupled to a first end of the shaft. The swab is saturated with the first ocular composition inside the wrapper. The first ocular composition comprises polyaminopropyl biguanide, 1,2 hexanediol and caprylyl glycol. A second ocular treatment kit comprises a first sealed wrapper and a second sealed wrapper. The first sealed wrapper contains an applicator while the second sealed wrapper contains a second ocular composition. The second ocular composition comprises tea tree oil, tea tree oil, sea buckthorn oil and a medium chain triglyceride.

### CROSS-REFERENCE TO RELATED APPLICATION

This application is a continuation-in-part of U.S. patent application Ser. No. 16/105,726, filed on Aug. 20, 2018, which is a divisional of U.S. patent application Ser. No. 14/707,187, filed on May 8, 2015, which is a continuation-in-part of U.S. patent application Ser. No. 14/274,198, filed on May 9, 2014, the contents of which are incorporated by reference in their entireties.

### FIELD OF THE INVENTION

Applicators, ocular compositions and methods which are useful for cleansing the eyelid area and maintaining eyelid hygiene are disclosed.

### BACKGROUND OF THE INVENTION

The eyelids are important to ocular health because they protect the eyes from airborne contaminants, such as pollen, dust particles or other foreign bodies. The eyelids contain several glands including the lacrimal glands and the specialized form of the sebaceous glands, the meibomian glands, which produce layers of tear film that are critical for healthy eyes.

The eyelids are subject to problems like blepharitis, dry eyes and inflammation of the meibomian glands. Another complication is the infestation of the arachnid, Demodex folliculorum (Demodex mites). The Demodex mite infestation is common in humans; anecdotal evidence suggests that the mites can be found in ten percent of the eyelashes of healthy persons. The occurrence of the infestation may also be age related.

Demodex mites reside inside the sebaceous glands and hair follicles. They cause damage to the cell walls by sucking nutrients from the hair roots. They burrow into the skin, lay eggs, introduce bacteria, and infect the skin. Some of the symptoms of infestation include itching and inflammation of the eyelids. Additionally, there is evidence that the mites can also be one of the causes of the skin disease rosacea.

### SUMMARY OF THE INVENTION

According to an embodiment, a method for treating Anterior Blepharitis and eyelid debridement involves providing a first ocular treatment kit. The ocular treatment kit comprises one or more sealed wrappers. Each wrapper contains a single-use applicator and a first ocular composition. The applicator has a shaft and a swab. The swab is coupled to a first end of the shaft. The swab is immersed in the first ocular composition in the wrapper such that the swab is saturated with the first ocular composition inside the wrapper. The first ocular composition comprises polyaminopropyl biguanide, 1,2 hexanediol and caprylyl glycol. The method involves keeping the wrapper in an upright position and unsealing it. This is followed by gently removing the applicator from the wrapper with the swab facing downward. The applicator is removed from a top end of the wrapper. The top end may be imprinted with descriptive indicia, such as, “TOP” to identify the top surface. A first eye area of the patient is treated by applying the saturated swab over the patient's eyebrow, closed eyelids and eyelashes. The treatment is repeated for the second eye area by applying the saturated swab over the patient's eyebrow, closed eyelids and eyelashes. The method further involves rinsing each treated eye area with an eye wash or a saline wipe. The method further involves removing any additional debris from the treated first or second eye area. The treated eye areas can be cleansed daily for at least two weeks with the first ocular composition.

According to another embodiment, a method for treating Demodex mites, comprises providing a second ocular treatment kit. The ocular treatment kit has a first and a second sealed wrapper. The first sealed wrapper contains an applicator having a shaft and a swab. The swab is coupled to a first end of the shaft. The second sealed wrapper contains a second ocular composition. The second ocular composition comprises tea tree oil, tea tree oil, sea buckthorn oil and a medium chain triglyceride. The medium chain triglyceride can be caprylic/capric triglyceride. The method involves removing the applicator from a top end of the first wrapper with the swab facing downward. The top end of the first wrapper can be imprinted with descriptive wording. The second sealed wrapper is opened at a top end and keeping the second sealed wrapper in an upright position, the applicator is inserted into the second wrapper such that the swab is immersed in the second ocular composition. The top end of the second sealed wrapper can also be imprinted with descriptive wording. Further, the first wrapper can be imprinted with suitable indicia to indicate that it contains the applicator while the second wrapper can be imprinted to indicate that it contains the second ocular composition. The saturated swab is used to treat a first eye area by applying it over a patient's eyebrow, closed eyelids and eyelashes. The treatment is repeated for the second eye area by applying the saturated swab over the patient's eyebrow, closed eyelids and eyelashes. The method further involves rinsing each treated eye area with an eye wash or a saline wipe. The method further involves removing any additional debris from the treated first or second eye area. The treated eye areas can be cleansed daily for at least two weeks with the second ocular composition. The method further involves removing any additional debris from the treated first or second eye area.

### BRIEF DESCRIPTION OF THE FIGURES

The features and advantages of certain embodiments will be more readily appreciated when considered in conjunction with the accompanying figures. The figures are not to be construed as limiting any of the preferred embodiments.

FIG. 1 depicts an applicator for treating or cleansing an ocular region according to one embodiment of the invention.

FIG. 2 depicts a first ocular treatment kit for treating or cleansing an eye area according to one embodiment of the invention.

FIG. 3 depicts a second ocular treatment kit for treating or cleansing an eye area according to another embodiment of the invention.

### DETAILED DESCRIPTION

As used herein, an eye area includes the eyes, eyelids and eyebrows or an area adjacent the eyes, eyelids and eyebrows.

Several compositions for cleansing or treating the ocular region are known in the art. These treatment compositions are usually applied to the eye area by means of the finger tips. Alternately, fabric wipes or pads may be impregnated with a treatment composition. However, it has been observed that the wipes may not retain an optimal amount of the treatment composition. The fabric pads or wipes may also be inconvenient or messy to handle. Consequently, the overall efficacy of the treatment may be impacted.

Accordingly, for all the reasons stated above, there is a need for a mechanism to deliver a desired amount of an ocular composition to the eye area. The mechanism should be convenient to use at home or in a doctor's office. The mechanism should facilitate a non-messy treatment of the ocular region.

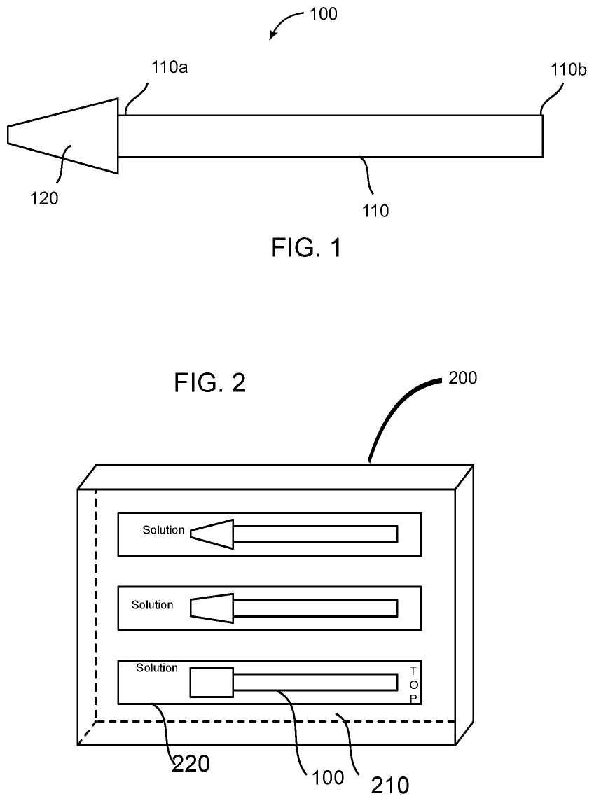

In one embodiment, an applicator and ocular composition suitable for both adjunctive eyelid therapy and hygiene maintenance is provided. Referring now to FIG. 1, in one embodiment of the invention, an applicator 100 for cleansing or treating an ocular region is provided. The applicator 100 includes a shaft 110 and a swab 120. The applicator 100 may be dimensioned for convenient handling by a user. The applicator is configured for a single use.

The shaft 110 may be manufactured of a suitable material known in the art. For example, the material may include, without limitation, plastic, polymer, resin, synthetic polymer, paper, wood, and a composite material. The shaft 110 includes a first end 110a and an opposing end 110b.

A swab 120 is bonded to or otherwise attached to the first end 110a. The swab 120 may comprise an absorbent material such as, foam, latex-free foam, cotton, fabric, sponge, fiber, felt, rayon, synthetic foam, synthetic sponge, textile and synthetic fiber. In one embodiment, the swab 120 may be made of 100PPI medical grade foam. The swab 120 is configured to absorb or be saturated with a desired or effective amount of a pre-determined ocular composition. The swab 120 is also capable of retaining the desired amount of the ocular composition.

The ocular composition is configured to cleanse the ocular region or treat the ocular region. The ocular composition may be a non-irritating liquid composition useful in cleansing the eyelids. The ocular composition may also be effective as an eyelid cleanser, as it has an antimicrobial effect. The use of non-irritating ingredients that also exhibit antimicrobial benefits in the ocular composition increases its cleansing ability. The ocular compositions may offer convenient combination therapy for improving overall eyelid hygiene and also providing for adjunctive eyelid therapy. Any suitable ocular composition can be used in conjunction with the applicator disclosed herein.

In one embodiment, the first ocular composition may include the eyelid scrub composition disclosed in U.S. Pat. No. 7,951,387 which is incorporated by reference herein in its entirety. For example, the ocular composition comprises polyaminopropyl biguanide, 1,2-hexanediol, and caprylyl glycol in combination with a pH stabilizing surfactant solution. Suitable surfactants to be used in the pH stabilizing surfactant solution include amphoteric surfactants, anionic surfactants, and nonionic surfactants. Suitable amphoteric surfactants include, but are not limited to alkyldimethyl betaines, alkylamido betaines, sulfobetaines, and imidazoline amphoterics. Suitable anionic surfactants include, but are not limited to fatty alcohol sulfates, alpha olein sulfonates, sulfosuccinates, sarcosinates, phosphate esters, and carboxylates. Suitable nonionic surfactants include, but are not limited to alkanolamids, ethoxylate amids, esters, aixylated alcohols, alkylpolyglucosides, amine oxides, sorbitan esters, and ethoxylates. The pH stabilizing surfactant solution comprises disodium cocoamphodiacetate, PEG-80 sorbitan laurate, decyl polyglucoside, and a modified Ringer's solution. Disdoium cocoamphodiacetate is an amphoteric surfactant. PEG-80 sorbitan monolaurate and decyl polyglucoside are both nonionic surfactants. In another embodiment, the composition, when mixed, comprises about 0.1 to 25 wt. % disodium cocoamphodiacetate, 0.1 to 10 wt. % PEG-80 sorbitan monolaurate, 0.2 to 10 wt. % decyl polyglucoside, and 60 to 98 wt. % modified Ringer's solution. The modified Ringer's solution comprises, sodium chloride, potassium chloride, calcium chloride, and water. Preferably, the water used is purified water. The modified Ringer's solution may also comprise 0.05 to 1.2 wt. % sodium chloride, 0.005 to 0.5 wt. % potassium chloride, 0.005 to 0.5 wt. % calcium chloride, and water. In still another embodiment, the modified Ringer's solution comprises about 0.7 wt. % sodium chloride, about 0.03 wt. % potassium chloride, about 0.033 wt. % calcium chloride, and purified water.

The first ocular composition can further comprise one or more moisturizers. Moisturizers are chemicals that prevent transepidermal water loss. Moisturizers may prevent water loss by forming a film over the skin to prevent water from evaporating from the skin. Alternatively, moisturizers comprise hydroscopic molecules that draw water from the air into the skin. Suitable moisturizers include, but are not limited to, methyl gluceth-20, sorbital, glycerine, propylene glycol, carboxylates, amino acids, glucoside derivatives, urea, lactates, and derivatives of pantothenic acid. Examples of derivatives of pantothenic acid include panthenol, D-panthenol, and D, L-panthenol.

The first ocular composition may also include a foam stabilizer. A foam stabilizer is a chemical which increases the lifetime of the foam. The foam stabilizer can be a polyethylene glycol diester of methyl glucose and a fatty acid. Suitable fatty acids include oleic acid, steric acid, lauric acid, caprylic acid, and capric acid. Suitably, the foam stabilizer is PEG-120 methyl glucose dioleate.

One specific embodiment of the first ocular composition comprises polyhexamethylene biguanide, 1,2-hexanediol, 1,2-octanediol, D-panthenol, cocoamphodiacetate disodium, polyoxyethylene-80 sorbitan monolaurate, decyl polyglucoside, methyl gluceth-20, and PEG-120 methyl glucose dioleate.

Another specific embodiment of the first ocular composition comprises about 0.02 wt. % to about 0.3 wt. % PHMB, about 0.05 wt. % to about 2.0 wt. % 1,2-hexanediol, about 0.05 wt. % to about 2.0 wt. % 1,2-octanediol, about 0.1 wt. % to about 25 wt. % cocoamphodiacetate disodium, about 0.1 wt. % to about 10 wt. % polyoxyethylene 80 sorbitan monolaurate, about 0.2 wt. % to about 10 wt. % decyl polyglucoside, and about 60 wt. % to about 98 wt. % Modified Ringer's Solution.

Yet another specific embodiment of the first ocular composition comprises about 0.04 wt. % polyhexamethylene biguanide, about 0.2 wt. % 1,2-hexanediol, about 0.2 wt. % 1,2-octanediol, about 0.2 wt. % D-panthenol, about 0.215 wt. % cocoamphodiacetate disodium, about 4.032 wt. % polyoxyethylene 80 sorbitan monolaurate, about 0.275 wt. % decyl polyglucoside, about 4.3 wt. % methyl gluceth-20, about 0.6 wt. % PEG-120 methyl glucose dioleate, about 87.985 wt. % Modified Ringer's Solution, and water.

Zinc salts are astringents which cause skin to tighten. The skin around the ocular area is more sensitive that other areas of skin. The inclusion of a zinc salt is a composition may be undesirable as its astringent property would make the composition more irritating to the eyelid area. Therefore, in one embodiment, the first ocular composition comprises PHMB, 1,2-hexanediol, 1,2-octanediol, and a pH stabilizing surfactant solution, but is also essentially free of zinc salts. Examples of zinc salts include zinc acetate, zinc lactate, zinc gluconate, zinc citrate, zinc butyrate, and zinc sterate.

In an embodiment of the invention, a first ocular treatment kit 200 is disclosed. The ocular treatment kit 200 can be configured for debridement of Anterior Blepharitis or Meibomian gland expression (or Posterior Blepharitis). As shown in FIG. 2, the kit 200 may include an enclosure or housing 210. The enclosure 210 may be a box or container made of paper, plastic or another suitable material known in the art. A plurality of applicators 100, as described earlier, may be contained within the enclosure 210. The kit 200 further comprises a wrapper or pouch 220. Each wrapper 220 contains an applicator 100 and the first ocular composition disclosed herein. The wrapper 220 maybe labelled with the wording to indicate a top and bottom surface. For instance, as shown in FIG. 2, the wrapper 220 is labeled with the word “TOP”. The enclosure 210 may be imprinted with information on proper usage of the applicator.

In one or more embodiments, the kit 200 is assembled by holding the wrapper 220 in an upright position (that is, the end labeled “TOP” faces upward). This is followed by placing a desired amount of the first ocular composition inside the wrapper 220. The first ocular composition settles at the bottom end of the wrapper 220. The applicator 100 is then inserted in the wrapper such that the swab tip is away from the side labelled “TOP”. The swab tip is fully immersed in the first ocular composition which is settled at the bottom end of the wrapper.

The kit 200 may further include a specialized double-ended soft brush (not shown) for removing scurf and debris from the eyelids and eyelashes to facilitate debridement of Anterior Blepharitis.

In another embodiment, a method for treatment of Anterior Blepharitis or eyelid debridement is disclosed. The patient suffering from Anterior Blepharitis can be treated in a doctor's office, such as, an ophthalmologist's office. The method involves providing the kit 200 having the wrapper 220 (disclosed in FIG. 2) comprising the applicator 100 and the first ocular composition. The wrapper is opened at the top end or the end labeled “TOP”. The applicator can be gently removed from the wrapper by pulling it out upwards through the top. This ensures that the doctor (or other health care professional) does not touch the first ocular composition impregnated swab or any residual solution at the bottom of the wrapper. The swab impregnated with the first ocular composition is applied to a patient's eyebrow, closed eyelids and eyelashes. This is followed by rinsing the treated eye area with a suitable eye wash, such as, OCuSOFT® Eye Wash or saline wipes. The process is repeated for the other eyebrow, eyelids and eyelashes followed by rinsing the second eye area with the eye wash or saline wipes. In an embodiment, a specialized brush can be used to remove any residual scurf or debris in the area around each eye. The method further involves a post-treatment maintenance program. This can involve cleansing the eyelid area with the first ocular composition for at least two weeks following the treatment. For instance, the patient can cleanse the eyelid area with single use pre-moistened pads or wipes that are impregnated with the first ocular composition.

In yet another embodiment, a second ocular composition is provided. The second ocular composition may include a mixture of tea tree oil, sea buckthorn oil and a medium chain triglyceride. Such a composition has been found effective in treating Demodex mites. The composition has been disclosed in U.S. Pat. Pub. No. 20120121694, the contents of which have been incorporated by reference herein. The medium chain triglyceride may comprise caprylic/capric triglyceride or any other suitable dermatological carrier for the tea tree oil and sea buckthorn oil. For example, in one or more embodiments, the second ocular composition may comprise about 45% to about 55% tea tree oil, about 15% to about 25% sea buckthorn oil, and about 25% to about 35% caprylic/capric triglyceride. In another embodiment, the second ocular composition may include 50% tea tree oil, 20% sea buckthorn oil, and 30% caprylic/capric triglyceride.

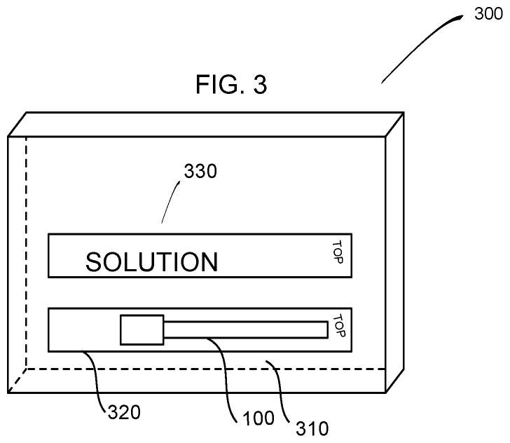

In yet another embodiment of the invention, a second ocular treatment kid 300 is provided. The ocular treatment kit 300 can be configured for treatment of Demodex. As shown in FIG. 3, the kit 300 may include an enclosure or housing 310. The enclosure 310 may be made of paper, plastic or another suitable material known in the art. A plurality of applicators 100, as described earlier, may be contained within the enclosure 310. The kit 300 further comprises a first wrapper or pouch 320. The first wrapper 320 contains an applicator 100. The first wrapper 320 may be labelled with wording to indicate a top and bottom surface. For instance, as shown in FIG. 3, the wrapper 320 can be imprinted with the word “TOP”. The first wrapper may also be imprinted with suitable wording to indicate that it contains the applicator. The kit 300 further includes a second wrapper 330. The second wrapper 330 is configured to receive a desired amount of the second ocular composition. The second wrapper 330 may be imprinted with wording to indicate a top and bottom surface. For instance, as shown in FIG. 3, the second wrapper 330 is labeled with the word “TOP”. The second wrapper may also be imprinted with suitable wording to indicate that it contains the second ocular composition. The enclosure 310 may be imprinted with information on proper usage of the applicator and the second ocular composition.

In one or more embodiments, the kit 300 is assembled by holding the first wrapper 320 in an upright position (that is, the end labeled “TOP” faces upward). The applicator 100 is then inserted in the first wrapper 320 such that the swab tip is away from the side labelled “TOP”. A desired amount of the second ocular composition is placed inside the second wrapper 330.

The kit 300 may further include a specialized double-ended soft brush (not shown) for removing scurf and debris from the eyelids and eyelashes.

The kit 300 may further include a pair of tweezers (not shown) for removing scurf and debris from the eyelids and eyelashes. The tweezers may have a cone tip and may be manufactured from plastic.

The kit 300 may further include one or more sterile saline wipes. The wipes may be individually wrapped to provide safe and gentle cleansing of the eye area while eliminating the risk of cross-contamination.

In another embodiment, a method for treating a patient having Demodex mites is disclosed. The patient suffering from the Demodex mites can be treated in a doctor's office, such as, an ophthalmologist's office. The method involves providing the kit 300 having the first and second wrappers 320, 330 (disclosed in FIG. 2). The first wrapper 320 is opened at the top end or the end labeled “TOP”. The applicator can be gently removed from the first wrapper by pulling it out upwards through the top. The second wrapper 330 is held in an upright position and the applicator 100 is inserted into the second wrapper such that the swab tip of the applicator is saturated or impregnated with the second ocular composition contained in the second wrapper 330. The swab impregnated with the second ocular composition is applied to a patient's eyebrow, closed eyelids and eyelashes. This is followed by rinsing the treated eye area with a suitable eye wash, such as, OCuSOFT® Eye Wash or saline wipes. The process is repeated for the other eyebrow, eyelids and eyelashes followed by rinsing the second eye area with the eye wash or saline wipes. The method further involves a post-treatment maintenance program. This can involve cleansing the eyelid area with the second ocular composition for at least two weeks following the treatment. For instance, the patient can cleanse the eyelid area with single use pre-moistened pads or wipes that are impregnated with the second ocular composition.

Although various exemplary ocular compositions have been described herein, the compositions disclosed herein are not intended to be limiting. In fact, any composition that can be used to cleanse or treat the eyes, eyelids, or an ocular region, may be used in accordance with the various embodiments of the invention.

It should be understood that, as used herein, “first,” “second,” etc., are arbitrarily assigned and are merely intended to differentiate between two or more treatment kits, compositions etc., and does not indicate any particular composition or sequence. Furthermore, it is to be understood that the mere use of the term “first” does not require that there be any “second,” and the mere use of the term “second” does not require that there be any “third,” etc.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is, therefore, evident that the particular illustrative embodiments disclosed above may be altered or modified and all such variations are considered within the scope and spirit of the present invention. While kits, compositions and methods are described in terms of “comprising,” “containing,” or “including” various components or steps, the kits, compositions and methods also can “consist essentially of” or “consist of” the various components and steps. Whenever a numerical range with a lower limit and an upper limit is disclosed, any number and any included range falling within the range is specifically disclosed. In particular, every range of values (of the form, “from about a to about b,” or, equivalently, “from approximately a to b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an”, as used in the claims, are defined herein to mean one or more than one of the element that it introduces. If there is any conflict in the usages of a word or term in this specification and one or more patent(s) or other documents that may be incorporated herein by reference, the definitions that are consistent with this specification should be adopted.

We claim:

1. A method for treating Anterior Blepharitis and eyelid debridement, comprising:
providing a first ocular treatment kit, the first ocular treatment kit comprising:
one or more sealed wrappers, each wrapper containing an applicator and a first ocular composition, the applicator comprising:
a shaft; and
a swab, wherein the swab is coupled to a first end of the shaft, and wherein the swab is saturated with the first ocular composition inside the wrapper;
wherein the first ocular composition comprises polyaminopropyl biguanide, 1,2 hexanediol and caprylyl glycol;
removing the applicator from the wrapper with the swab facing downward; and
treating a first eye area by applying the saturated swab over the patient's eyebrow, closed eyelids and eyelashes.

2. The method according to claim 1, further comprising treating a second eye area by applying the saturated swab over the patient's eyebrow, closed eyelids and eyelashes.

3. The method according to claim 2, further comprising rinsing each treated eye area with an eye wash or a saline wipe.

4. The method according to claim 1, further comprising cleansing the treated eye areas daily for at least two weeks with the first ocular composition.

5. The method according to claim 1, further comprising keeping the wrapper in an upright position and unsealing the wrapper prior to removing the applicator.

6. The method according to claim 5, wherein the applicator is removed from a top end of the wrapper, and wherein the top end of the wrapper is labeled with descriptive wording.

7. The method according to claim 2, further comprising removing any additional debris from the treated first or second eye area.

8. A method for treating Demodex mites, comprising:
providing a second ocular treatment kit, the second ocular treatment kit comprising:
a first sealed wrapper, the first sealed wrapper containing an applicator, the applicator comprising:
a shaft; and
a swab, wherein the swab is coupled to a first end of the shaft;
a second sealed wrapper, the second sealed wrapper containing a second ocular composition, wherein the second ocular composition comprises tea tree oil, tea tree oil, sea buckthorn oil and a medium chain triglyceride;
removing the applicator from the first wrapper with the swab facing downward;
opening the second sealed wrapper at a top end and keeping the second sealed wrapper in an upright position, inserting the applicator into the second wrapper such that the swab is immersed in the second ocular composition; and
saturating the swab and treating a first eye area by applying the saturated swab over the patient's eyebrow, closed eyelids and eyelashes.

9. The method according to claim 8, further comprising treating a second eye area by applying the saturated swab over the patient's eyebrow, closed eyelids and eyelashes.

10. The method according to claim 9, further comprising rinsing each treated eye area with an eye wash or a saline wipe.

11. The method according to claim 8, further comprising cleansing the treated eye areas daily for at least two weeks with the second ocular composition.

12. The method according to claim 1, further comprising keeping the first wrapper in an upright position and unsealing the first wrapper prior to removing the applicator.

13. The method according to claim 12, wherein the applicator is removed from a top end of the first wrapper, and wherein the top end of the first wrapper is labeled with descriptive wording.

14. The method according to claim 8, wherein the top end of second wrapper is labeled with descriptive wording.

15. The method according to claim 9, further comprising removing any additional debris from the treated first or second eye area.

16. A first ocular treatment kit, comprising:
one or more sealed wrappers, each wrapper containing an applicator and a first ocular composition, the applicator comprising:
a shaft; and
a swab, wherein the swab is coupled to a first end of the shaft, and wherein the swab is saturated with the first ocular composition inside the wrapper;
wherein the first ocular composition comprises polyaminopropyl biguanide, 1,2 hexanediol and caprylyl glycol.

17. A second ocular treatment kit, comprising:
a first sealed wrapper, the first sealed wrapper containing an applicator, the applicator comprising:
a shaft; and
a swab, wherein the swab is coupled to a first end of the shaft; and
a second sealed wrapper, the second sealed wrapper containing a second ocular composition, wherein the second ocular composition comprises tea tree oil, tea tree oil, sea buckthorn oil and a medium chain triglyceride.
